(19) United States
(12) Patent Application Publication
(10) Pub. No.: US 2025/0022594 A1
(43) Pub. Date: Jan. 16, 2025

(54) DYNAMIC PROGRAM RECOMMENDATION WITH REDUCED NETWORK CONNECTIONS

(71) Applicant: XanthosHealth, Inc., Saint Paul, MN (US)

(72) Inventors: David Haynes, II, Brooklyn Park, MN (US); Pinar Karaca Mandic, Saint Paul, MN (US)

(21) Appl. No.: 18/770,885

(22) Filed: Jul. 12, 2024

Related U.S. Application Data
(60) Provisional application No. 63/513,584, filed on Jul. 14, 2023.

(51) Int. Cl. G16H 40/67; G16H 10/60

(52) U.S. Cl. CPC G16H 40/67; G16H 10/60

(57) ABSTRACT

A method includes receiving patient attributes of a patient through a single secure network connection and comparing the patient attributes received through the single secure network connection to eligibility requirements of multiple community-based organization programs to identify a plurality of community-based organization programs that the patient qualifies for enrollment in. A first community-based organization provides a first community-based organization program of the plurality of community-based organization programs and a second community-based organization provides a second community-based organization program of the plurality of community-based organization programs. The plurality of community-based organization programs that the patient qualifies for enrollment in are returned through one or both of the single secure network connection and a second secure network connection.

### CROSS-REFERENCE TO RELATED APPLICATION

The present application is based on and claims the benefit of U.S. provisional patent application Ser. No. 63/513,584, filed Jul. 14, 2023, the content of which is hereby incorporated by reference in its entirety.

This invention was made with government support under 75N91021C00044 awarded by the National Institutes of Health, National Cancer Institute. The government has certain rights in the invention.

### BACKGROUND

Social determinants of health (SDoH) such as housing instability, food insecurity, financial burden among others shape the health and well-being of individuals, create health inequities, and drive a large portion of avoidable adverse health outcomes and healthcare costs. When a patient with such needs is placed on a course of treatment by a medical care provider, the SDoH of the patient can reduce the effectiveness of that treatment. Because these needs are outside of the scope assistance that a medical care professional is able to provide, the patient must seek assistance elsewhere to address these needs.

Community-based organizations provide programs to help people with various needs such as housing, food, employment, transportation, safety, substance use, mental health and financial support.

The discussion above is merely provided for general background information and is not intended to be used as an aid in determining the scope of the claimed subject matter. The claimed subject matter is not limited to implementations that solve any or all disadvantages noted in the background.

### SUMMARY

A method includes receiving patient attributes of a patient through a single secure network connection and comparing the patient attributes received through the single secure network connection to eligibility requirements of multiple community-based organization programs to identify a plurality of community-based organization programs that the patient qualifies for enrollment in. A first community-based organization provides a first community-based organization program of the plurality of community-based organization programs and a second community-based organization provides a second community-based organization program of the plurality of community-based organization programs. The plurality of community-based organization programs that the patient qualifies for enrollment in are returned through one or both of the single secure network connection and a second secure network connection.

In accordance with a further embodiment, a server includes a network interface and a processor configured to perform steps. The steps include establishing a single secure network connection to an electronic device through the network interface and receiving patient attributes of a patient through the single secure network connection. The patient attributes received through the single secure network connection are compared to eligibility requirements of multiple community-based organization programs to identify a plurality of community-based organization programs that the patient qualifies for enrollment in. A first community-based organization provides a first community-based organization program of the plurality of community-based organization programs and a second community-based organization provides a second community-based organization program of the plurality of community-based organization programs. The processor establishes a second network connection to the electronic device through the network interface and returns the plurality of community-based organization programs that the patient qualifies for enrollment in through one or both of the single secure network connection and the second network connection.

In accordance with a still further embodiment, a method includes receiving a single API call providing patient attributes of a patient and comparing the patient attributes received through the single API call to eligibility requirements of multiple community-based organization programs to identify a plurality of community-based organization programs that the patient qualifies for enrollment in. A first community-based organization provides a first community-based organization program of the plurality of community-based organization programs and a second community-based organization provides a second community-based organization program of the plurality of community-based organization programs. The plurality of community-based organization programs that the patient qualifies for enrollment in are returned in response to a second API call.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a block diagram of a system in accordance with one embodiment.

FIG. 2 is a flow diagram of a method of enrolling patients in community-based organization programs.

FIG. 3 is a flow diagram of a method of adding a program to a server.

FIG. 4 is a user interface used to add a need, capacity, start date and end date of a program.

FIG. 5 is a user interface for adding program eligibility criteria for a program.

FIG. 6 is a flow diagram of a method of obtaining patient attributes in a secure manner.

FIG. 7 is a user interface used to acquire patient attributes from a patient.

FIG. 8 is an example user interface used in a needs assessment.

FIG. 9 is an example user interface used in a needs assessment.

FIG. 10 is a flow diagram for ranking programs for a patient.

FIG. 11 is an example user interface returned to a patient to allow a patient to request enrollment in a program

FIG. 12 is a flow diagram of a method for referring programs to a patient.

FIG. 13 is an example of a user interface used to refer a program to a patient.

FIG. 14 is a simplified block diagram of a system in accordance with one embodiment.

FIG. 15 is a block diagram of a computing device that can be used as a server and or electronic device in the various embodiments.

### DETAILED DESCRIPTION

There are a large number of community-based organizations, which each have their own computer system to provide information about their program and to receive requests to enroll in their programs. As part of the enrollment request, organizations request information about certain attributes of the person requesting enrollment.

When a person has several different needs, the distributed nature of the computer system requires the person to make multiple network connections to multiple different computing systems to enter their attributes. Establishing multiple network connections and sending the same attributes across those connections is computationally inefficient and therefore degrades the computer network by sending the same information multiple times over the computer network. This architecture also increases the chance that the person's attributes will be wrongfully acquired by a bad actor who “hacks” one of the network connections because each network connection provides a new opportunity for a hacker to acquire the information.

In addition, the computer systems of some community-based organizations require users to complete a needs survey to identify what needs the user has. This can also result in a user entering the same information multiple times across multiple different network connections resulting in inefficiencies and increasing the chances that the user's responses to these survey questions will be wrongfully acquired by a bad actor.

The embodiments described below provide an alternative computing architecture that improves the computer system by comparing a person's attributes to the eligibility criteria of multiple community-based organization programs while only requiring a single secure network connection to receive the attributes. By using only a single secure network connection, the embodiments improve the efficiency of the computing system and reduce the chances that the person's attributes will be obtained by bad actors. Thus, the embodiments improve both the efficiency and the security of the computing system.

Similarly, some embodiments below only require a user to complete a needs survey once instead of multiple times for multiple different community-based organizations. This improves the efficiency and security of the needs survey. In addition, only parties that the user has previously authorized may access the results of the survey. This provides more control to the user over the distribution of their current needs.

In addition, many of the programs have a maximum number of participants and when that maximum number is reached, the program is closed. In many embodiments described below, the number of participants enrolled in a program is kept track of and is compared to the maximum number of participants set for the program. When a program has reached its maximum capacity, the system will not return the program to the patient as a possible matching program. This improves the efficiency of the system by not returning data for programs that a person could not enroll in.

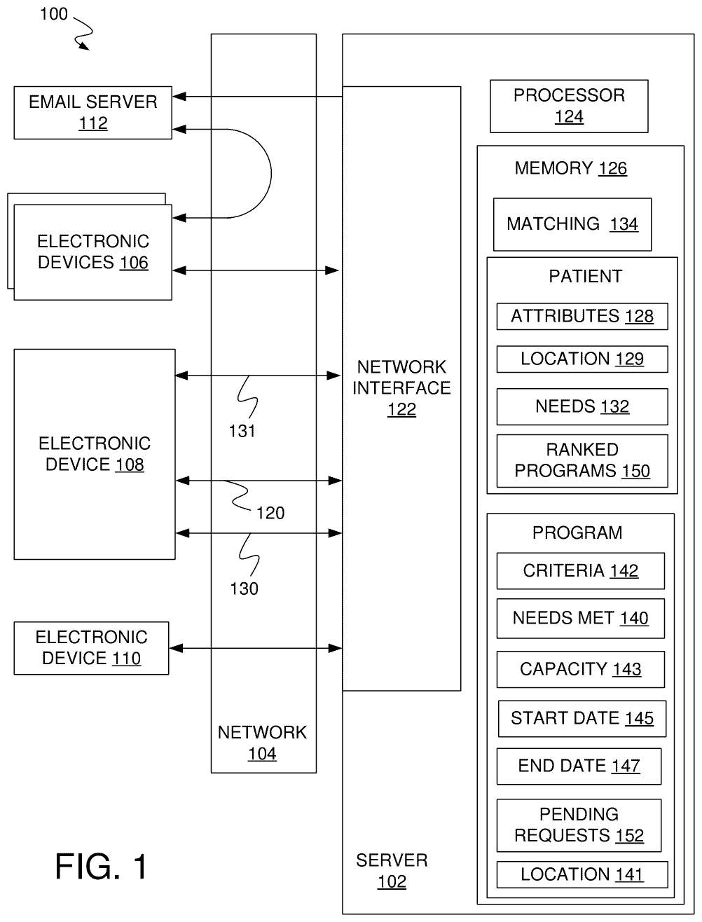

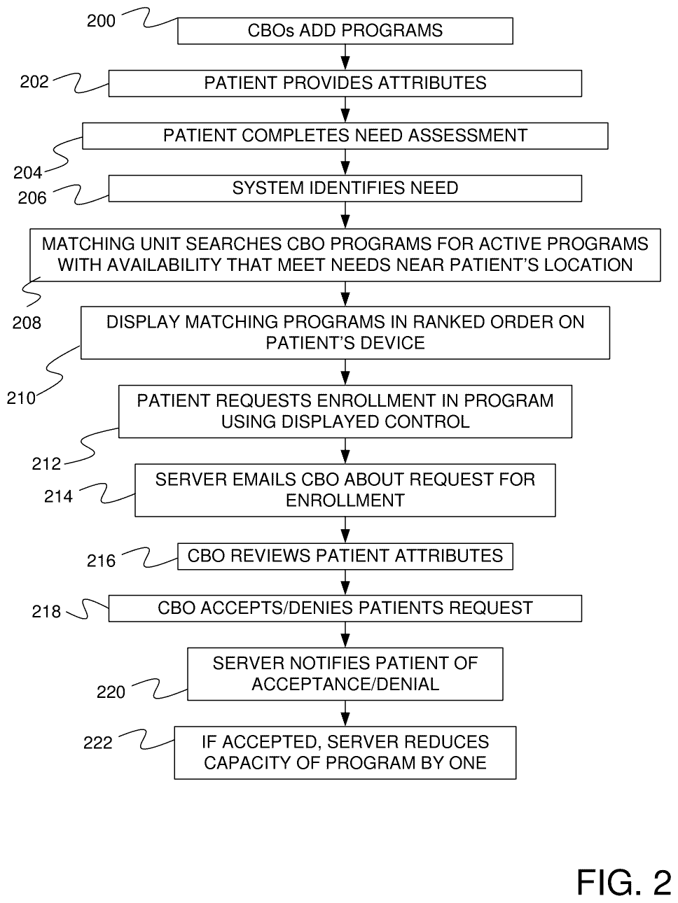

FIG. 1 provides a block diagram of the computer architecture and FIG. 2 provides a flow diagram of a method of using the computer architecture of FIG. 1. In the embodiments below, the person needing services is referred to as a patient. However, in other embodiments, the person needing services is not a patient. The system and methods described below may be used with non-patients as well as patients. For example, the system and methods may be used with a patient representative such as a home caregiver or a social worker.

Computer system 100 of FIG. 1 includes a server 102, a network 104, electronic devices 106, 108 and 110 and an email server 112.

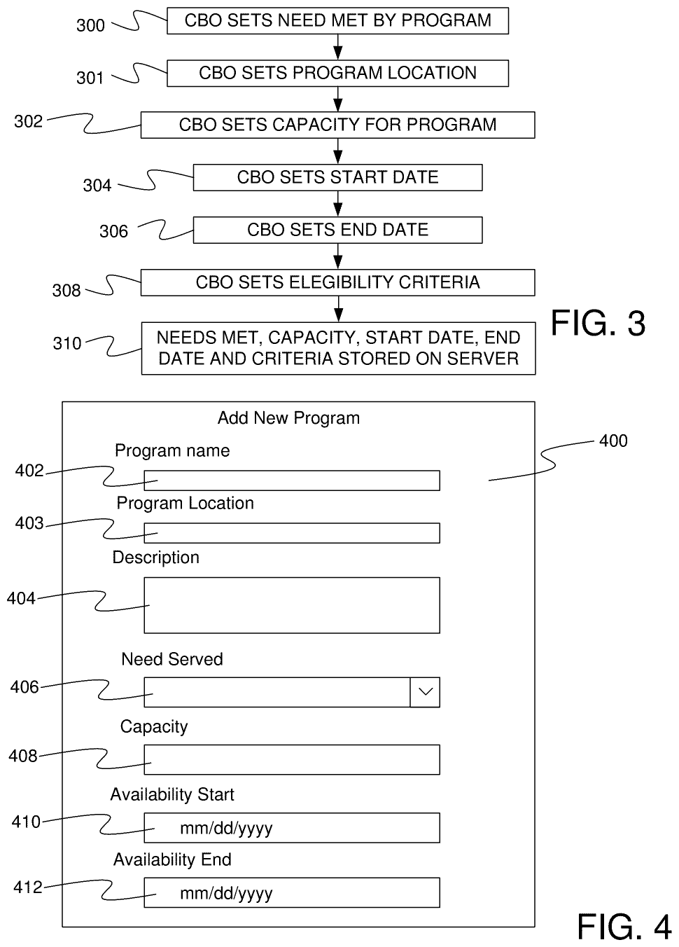

In step 200 of FIG. 2, multiple community-based organizations use respective electronic devices 106 to add community-based organization programs. FIG. 3 provides a flow diagram of a method performed in step 200 by a single community based organization. The method of FIG. 3 is performed using user interfaces displayed on a respective one of the electronic devices 106. These user interfaces may be retrieved from server 102 by making a request for the user interfaces or may be displayed on electronic device 106 by executing an application previously downloaded onto electronic device 106. An example of a first user interface 400 used to add a program is shown in FIG. 4.

As shown in FIG. 4, the community-based organization provides a program name 402 and a description of the program 404. At step 300, the community-based organization designates what need is served by the program using pull-down menu 406. Examples of needs include housing, food, transportation, safety, substance abuse counseling, mental health counseling and financial support, for example. Other needs may be provided in the pull-down menu. At step 301, the community-based organization sets a location for the program in location input 403. By acquiring the location for the program, the efficiency of the system is improved by not returning programs to a patient if the programs are remote from the patient. At step 302, the community-based organization sets the capacity/maximum number of participants that can be enrolled in the program using capacity input 408. By acquiring the capacity from the community-based organization, server 102 is able to determine when a program is unable to enroll any more people. As noted below, when the maximum number of enrollments has been reached, the program will no longer be returned as a possible program for enrollment. This improves the efficiency of the system by not returning data for programs that a person could not enroll in.

At steps 304 and 306, the community-based organization sets the start date and end date, respectively for the program using start date box 410 and end date box 412. By acquiring the start/end dates for the program, server 102 is able to determine when a program is yet to be available or is no longer available and thereby is able to determine which programs to return when a person is searching for programs to avoid returning programs that are not currently available. This improves the efficiency of the system by not returning data for programs that a person could not enroll in.

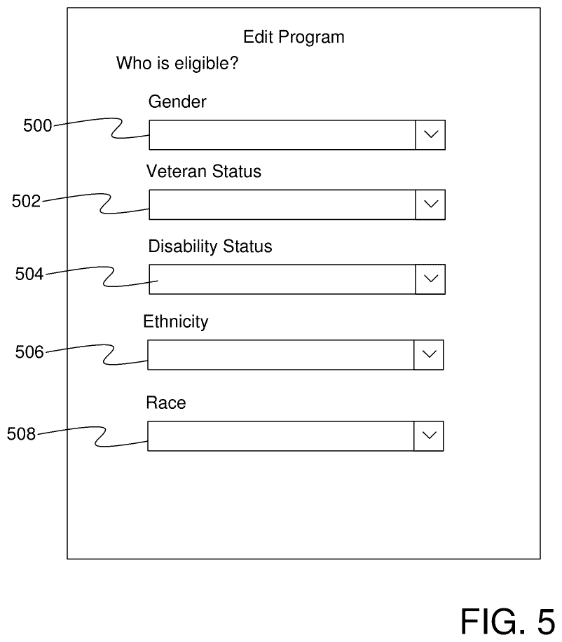

FIG. 5 provides a second user interface that is used in step 308 of FIG. 3 to allow the community-based organization to set eligibility criteria for the program. The eligibility criteria can include, for example, gender 500, veteran status 502, disability status 504, ethnicity 506 and race 508. For some community-based organizations, the eligibility criteria are strict criteria in which a person must fit within the criteria in order to be enrolled in the program. In other embodiments, the eligibility criteria are preferences but not requirements for entering the program and the community-based organization will at times admit people who do not meet all of the criteria for the program.

At step 310, electronic device 106 establishes a secure connection with server 102 through network 104 and transmits the program name, the need served by the program, the location for the program the capacity, start date and end date and the eligibility criteria to server 102. Server 102 stores this information in a memory 126 as need met 140, location 141, capacity 143, start date 145, end date 147 and criteria 142 within one or more records associated with the program.

The method of FIG. 3 is performed by multiple different community-based organizations to add multiple different programs. Thus, different programs in server 102 are provided by different community-based organizations.

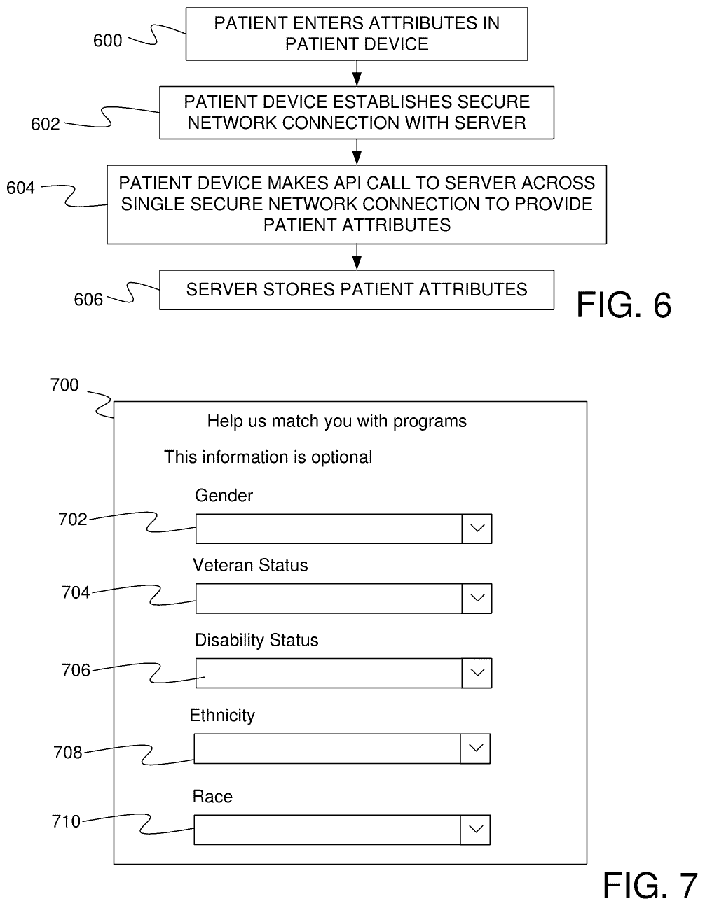

Returning to FIG. 2, after the community-based organization sets the program details at step 200, or before the community-based organization sets the program details at step 200, a patient provides attributes for themselves. FIG. 6 provides a flow diagram of a method performed in step 202.

In step 600 of FIG. 6, the patient enters patient attributes in electronic device 108. In accordance with one embodiment, the attributes are entered using a user interface, such as user interface 700 of FIG. 7. In accordance with embodiment, the patient attributes include the patient's location and attributes that correlate to the eligibility criteria such that each eligibility criterion appears as a patient attribute. Thus, in user interface 700, the patient attributes include gender 702, veteran status 704, disability status 706, ethnicity 708 and race 710, which correspond to gender 500, veteran status 502, disability status 504, ethnicity 506 and race 508 of the community-based organization user interface of FIG. 5. Although, criteria associated with one embodiment are shown in FIGS. 5 and 7, in other embodiments other criteria may be used in addition to or in place of the criteria shown.

At step 602, electronic device 108 establishes a secure network connection 120 to server 102. The secure network connection is made through network interface 122 of server 102. At step 604, electronic device 108 makes an API call to server 102 through this single secure network connection and provides the patient attributes as part of the API call. In accordance with the various embodiments, this is the only secure network connection that is established to provide the patient's attributes thereby improving the efficiency and the security of the computing system. Further, only a single API call is used to transmit the attributes. At step 606, a processor 124 in server 102 stores the patient attributes in memory 126 as attributes 128 and the patient's location as location 129.

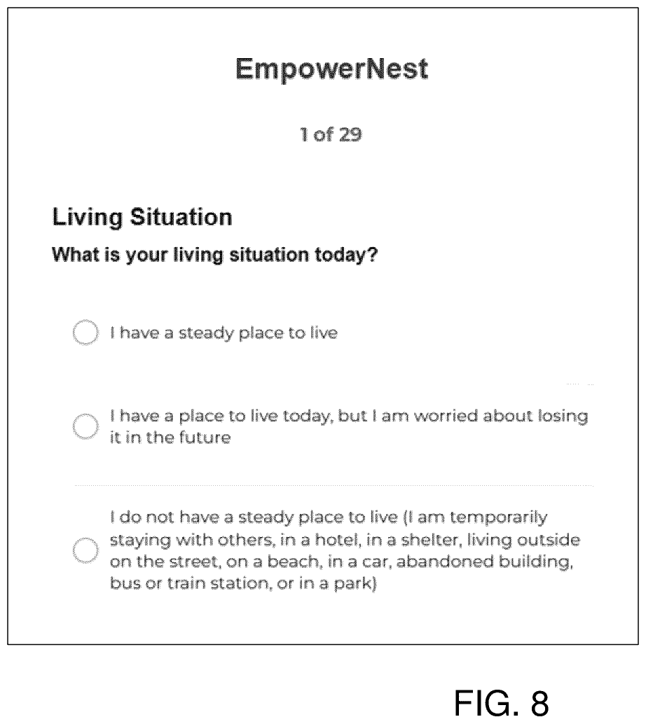

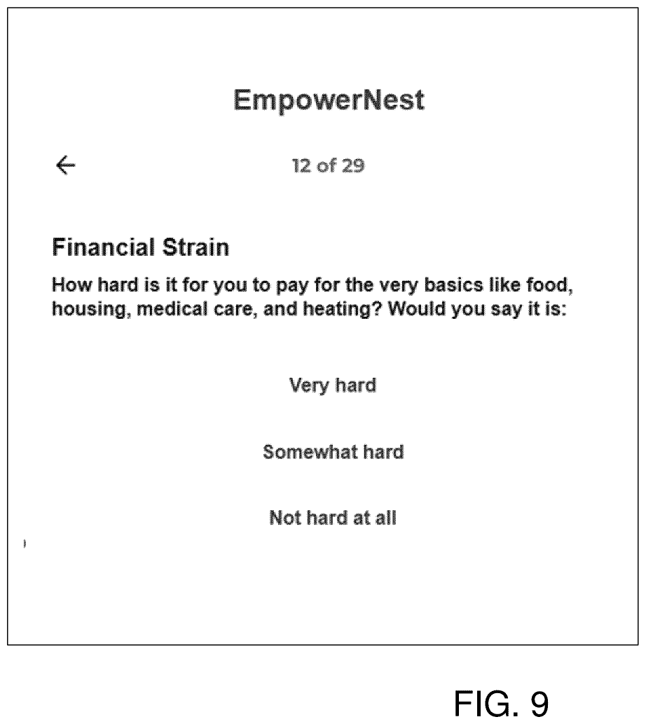

At step 204, the patient executes a needs assessment on electronic device 108 consisting of a series of user interfaces that request information from the patient to identify the needs of the patient. Examples of such user interfaces are shown in FIGS. 8 and 9. In FIG. 8, the patient is asked to describe their current living situation and in FIG. 9, the patient is asked to describe the level of financial strain they are experiencing. The user interfaces provided during the needs assessment can either be downloaded from server 102 by electronic device 108 or can be generated by an application previously downloaded to electronics device 108. The answers to the assessment are provided through a second secured network connection 130 to sever 102.

In step 206, server 102 uses the information from the needs assessment to identify needs of the patient. Server 102 then stores these as needs 132 in memory 126. In accordance with one embodiment, server 102 sends the identified needs to electronic device 108 and allows the user to remove any identified need. If the user removes an identified need, patient needs 132 is updated to remove that need.

Although server 102 is shown above to identify the needs, in other embodiments, electronic device 108 executes one or more functions to identify the needs from the patient's responses to the assessment. After the electronic device has identified such needs, it forwards those needs to server 102, which stores them as patient needs 132.

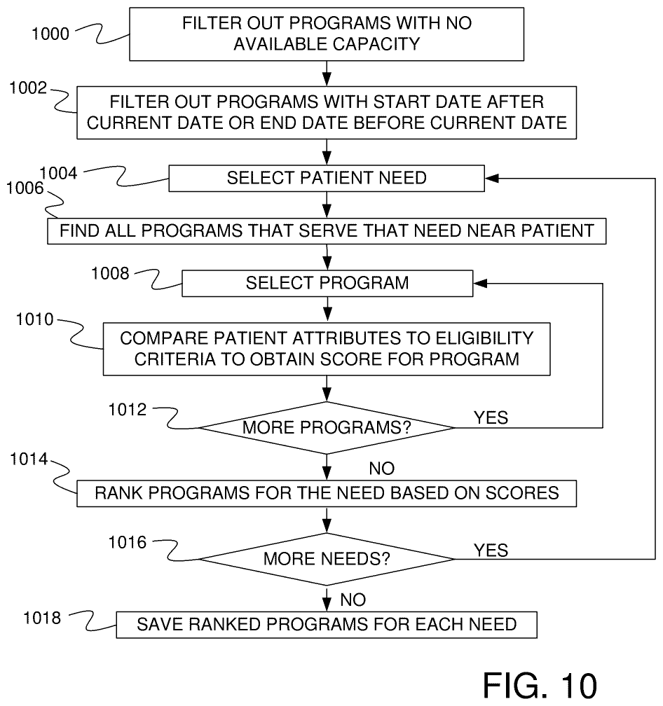

At step 208, a matching program 134 in memory 126 is executed by processor 124 of server 102 to search for programs that match the patient's attributes 128 and needs 132. FIG. 10 provides a flow diagram of a method performed in step 208.

In step 1000 of FIG. 10, matching program 134 filters out programs that have no remaining capacity 143. With each enrollment in a program, server 102 reduces the value of capacity 143 by one. As such, at step 1000, matching program 134 checks capacity 143 to see if there is any remaining capacity. If there is no remaining capacity for the program, the program will not be returned to the patient.

At step 1002, matching program 134 filters out programs with a start date after the current date or an end date before the current date. This removes programs that are not available yet or are no longer available and prevents such programs from being returned to the patient.

At step 1004, matching program 134 selects a patient need 132 and at step 1006, finds all programs with a location 141 near the patient's location 129 that service that need using needs met 140 of each program. By returning only programs near the patient's location, the system improves the efficiency of the system by only returning programs that would be relevant to the patient.

At step 1008, matching program 134 selects one of the programs that services the patients need and at step 1010, compares the patient's attributes 128 to the eligibility criteria 142 of the program to obtain a score for the program. In particular, for every eligibility criterion that the patient satisfies, the program is given a point and the total number of eligibility criteria that the patient satisfies provides the score for the program.

At step 1012, matching program 134 determines if there are more programs that service the patient's need. If there are more programs, the process returns to step 1008 and another program is selected. Step 1010 is repeated for the new program to obtain a score for the new program. When all of the programs that serve the need have been scored at step 1012, the programs are ranked for the need at step 1014. In particular, the programs with higher scores are ranked higher than the programs with lower scores. Although one scoring system has been described above, those skilled in the art will recognize that additional or alternative items can contribute to the score associated with a program.

At step 1016, matching program 134 determines if there are more patient needs. If there are more patient needs, the process returns to step 1004 to select the next patient need. Steps 1006-1014 are then repeated. When all of the patient's needs have been used to search for programs, the ranked programs for each need are saved at step 1018 as ranked programs 150 in FIG. 1.

At step 210 of FIG. 2, the matching programs are displayed in ranked order on electronic device 108. In accordance with one embodiment, electronic device 108 establishes a secure network connection 131 and makes an API call across secure network connection 131 to request all programs that have been matched to the patient. This API call can be made in response to a patient's action on electronic device 108 or may be generated periodically or in response to some other event by an application executing on electronic device 108.

In accordance with one embodiment, to view the matching programs, the patient first selects a need displayed on electronic device 108. The matching programs are then displayed in ranked order in a user interface for that need.

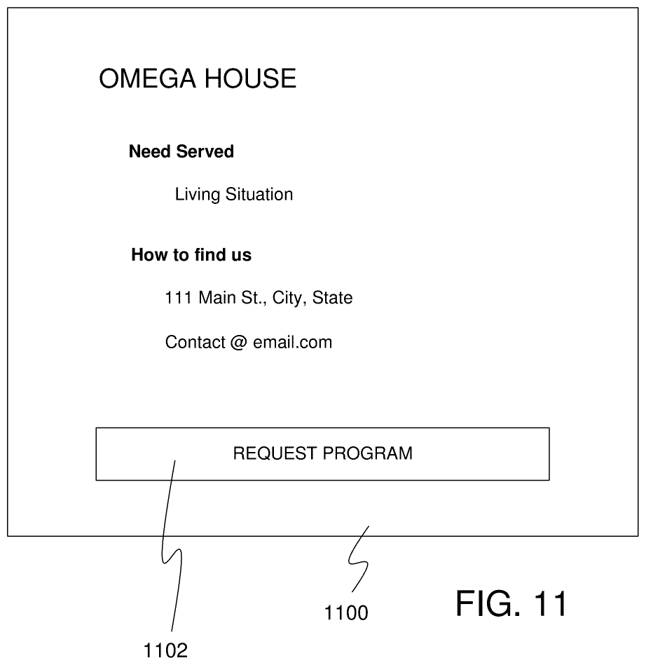

At step 212, the patient requests enrollment in a program using a control displayed for the program when the user selects the program from the ranked list. FIG. 11 shows an example of a user interface 1100 showing a control 1102 for requesting enrollment in a program. When the user selects control 1102, electronic device 108 sends a message to server 102 providing the patient identifier and the program identifier. Server 102 then sends an email through email server 112 to the community-based organization at step 214. In other embodiments, other communication channels are used to notify the community-based organization such as in-app notifications and text messaging. The email indicates that a patient is requesting enrollment in their program. Server 102 also updates a pending request list 152 to indicate that the patient has requested enrollment in the program.

At step 216, the community-based organization uses electronic device 106 to review the patient's attributes and at step 218, the community-based organization uses electronic device 106 to either accept or deny the patient enrollment into the program. At step 220, the patient is notified of either the acceptance or denial of enrollment into the program. If the patient is accepted into the program, server 102 automatically reduces the available capacity 143 for the program at step 222.

Further embodiments provide a mechanism for healthcare providers and community-based organizations to refer a program to a patient so as to encourage the patient to enroll in the program. In particular, a healthcare provider, social worker or community-based organization can refer a program to a patient.

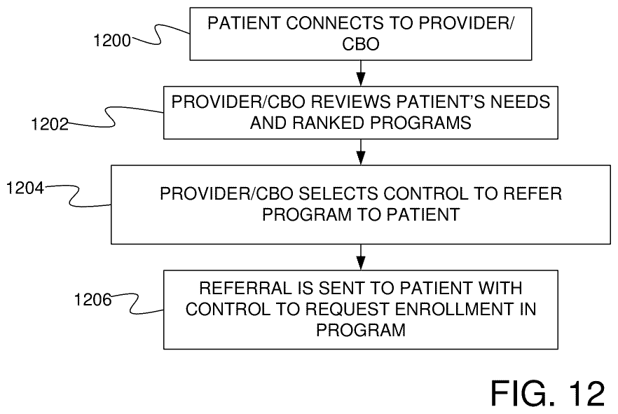

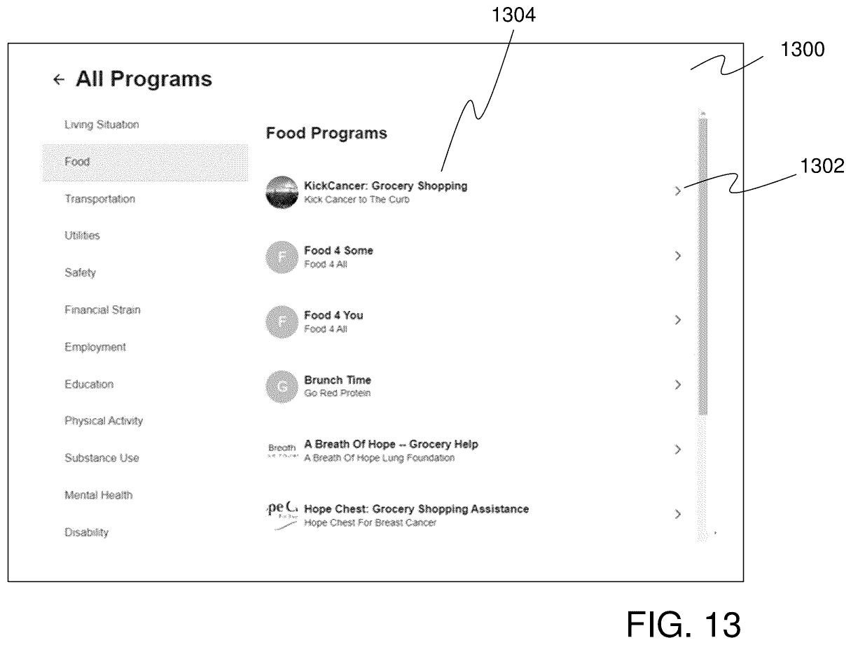

FIG. 12 provides a flow diagram of a method of referring programs to a patient from a secondary role such as a healthcare provider, social worker or community-based organization. In step 1200, the patient must first connect to the healthcare provider or community-based organization. Without this connection, the healthcare provider and community-based organization are prevented from viewing the patient's needs and are prevented from making a referral to the patient. At step 1202, the healthcare provider, social worker or community-based organization uses electronic device 110 to review the patient's needs and ranked matching programs for those needs. In particular, the provider can use a user interface, such as user interface 1300 of FIG. 13, to review the patient's needs and matching programs. At step 1204, the provider/social worker/organization selects a control on the user interface to refer the program to the patient. For example, a provider/social worker/organization can select control 1302 for program 1304 to show a referral control which can then be selected to refer the program to the patient.

At step 1206, server 102 receives the referral instruction from electronic device 110 and then sends a referral to electronic device 106 to refer the program to the patient. This referral will include a control in a description of the program that allows the patient to request enrollment in the program. The enrollment process is the same as described above.

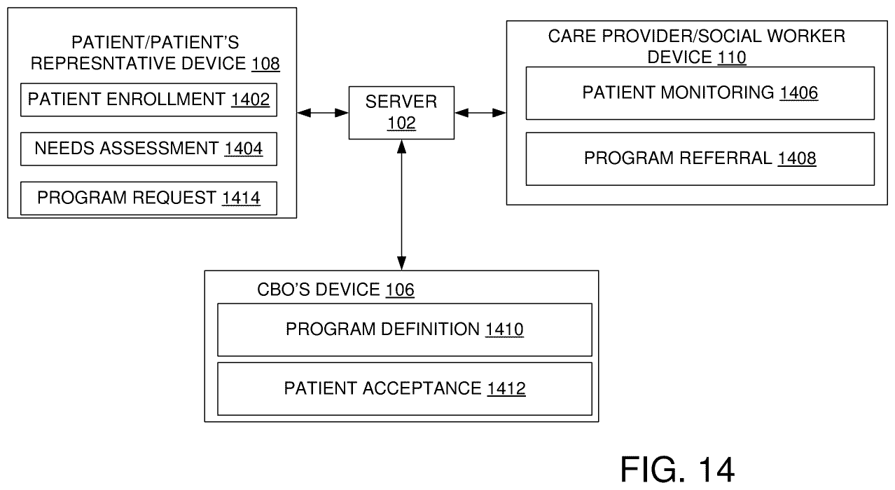

FIG. 14 provides a simplified block diagram showing the various connections between possible participant devices using the various embodiments described above. In FIG. 14, CBO's device 106 executes program definition 1410 to obtain the criteria and location of a program including the needs met by the program. CBO's device 106 provides the program's criteria, need and location to server 102. Patient/patient's representative device 108 executes patient enrollment 1402 to obtain the patient's attributes including the patient's location. Patient enrollment 1402 provides the patient attributes to server 102. Patient/patient's representative device 108 also executes needs assessment 1404 to identify the patient's needs. The patient's needs are provided to server 102.

Server 102 matches the patient's needs to programs that serve those needs that are located near the patient. Server 102 ranks the matching programs based on the programs' criteria and the patients attributes and returns the ranked programs to patient/patient's representative device 108. Patient/patient's representative device 108 executes program request 1414 to allow the patient/patient representative to view the ranked programs and to request enrollment in a program.

Requests for enrollment in a program are provided to server 102, which forwards the requests to CBO's device 106. CBO's device 106 executes patient acceptance 1412 to allow a CBO to indicate whether a patient has been accepted into a program. The acceptance is transmitted to server 102, which forwards the acceptance to patient/patient's representative device 108.

Care Provider/Social Worker device 110 executes patient monitoring 1406 to allow a care provider/social worker to view the needs of the patient and the programs that have been matched to the patient. In accordance with one embodiment, patient monitoring 1406 can only be executed for a patient who has authorized the care provider/social worker to view the patient's needs and programs. Care provider/social worker device 110 also executes program referral 1408, which allows a care provider/social worker to refer or recommend a particular program to the patient. When program referral 1408 refers a program, the referral is provided to server 102, which forwards the referral to patient/patient's device 108.

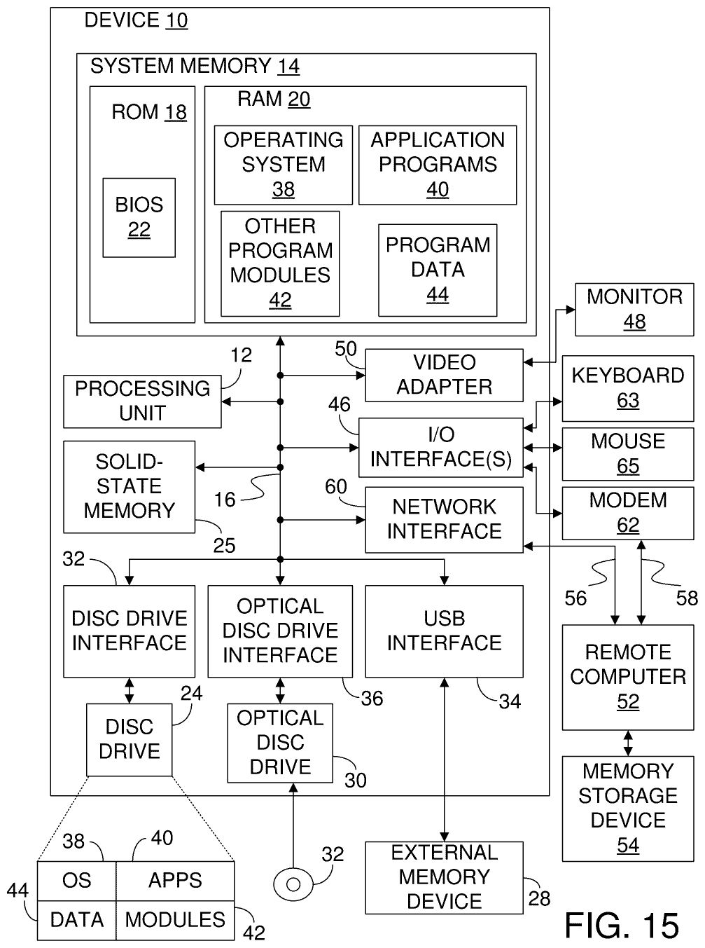

FIG. 15 provides an example of a computing device 10 that can be used to implement one or more of the servers and electronic devices discussed above. Computing device 10 includes a processing unit 12, a system memory 14 and a system bus 16 that couples the system memory 14 to the processing unit 12. System memory 14 includes read only memory (ROM) 18 and random-access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Computing device 10 further includes an optional hard disc drive 24, an optional external memory device 28, and an optional optical disc drive 30. External memory device 28 can include an external disc drive or solid-state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of the applications discussed above. Program data 44 may include any data used by the systems and methods discussed above.

Processing unit 12, also referred to as a processor, executes programs in system memory 14 and solid-state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 15. The network connections depicted in FIG. 15 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 15 are exemplary and other means for establishing a communications link between the computers, such as a wireless interface communications link, may be used.

Although the block diagram of FIG. 15 shows a computer, in other embodiments, one or more of electronic devices 106, 108 and 110 can be phones containing processors and network interfaces.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A method comprising:
through a single secure network connection, receiving patient attributes of a patient;
comparing the patient attributes received through the single secure network connection to eligibility requirements of multiple community-based organization programs to identify a plurality of community-based organization programs that the patient qualifies for enrollment in wherein a first community-based organization provides a first community-based organization program of the plurality of community-based organization programs and a second community-based organization provides a second community-based organization program of the plurality of community-based organization programs; and
returning the plurality of community-based organization programs that the patient qualifies for enrollment in through one or both of the single secure network connection and a second secure network connection.

2. The method of claim 1 further comprising;
receiving indications of needs of the patient; and
using the indications to select community-based organization programs that serve at least one of the needs such that comparing the patient attributes received through the single secure network connection to eligibility requirements is only performed on community-based organizations that serve at least one of the needs.

3. The method of claim 1 wherein returning the plurality of community-based organization programs comprises providing at least one control for requesting enrollment in one of the plurality of community-based organization programs.

4. The method of claim 3 further comprising:
providing an indication to a community-based organization that the patient has requested to enroll in one of the community-based organization's programs; and
providing updates to an electronic device indicating the current status of the request to enroll in one of the community-based organization's programs.

5. The method of claim 1 wherein each of the plurality of community-based organization programs has an end date and identifying the plurality of community-based organization programs comprises excluding community-based organization programs with an end date before a current date.

6. The method of claim 5 wherein for each community-based organization program, the end date is supplied by the community-based organization that provides the community-based organization program.

7. The method of claim 1 wherein each of the plurality of community-based organization programs has a maximum number of participants that can be enrolled in the program and wherein identifying the plurality of community-based organization programs comprises excluding community-based organization programs that have enrolled the respective maximum number of participants.

8. A server comprising:
a network interface; and
a processor configured to perform steps comprising:
establishing a single secure network connection to an electronic device through the network interface;
through the single secure network connection, receiving patient attributes of a patient;
comparing the patient attributes received through the single secure network connection to eligibility requirements of multiple community-based organization programs to identify a plurality of community-based organization programs that the patient qualifies for enrollment in wherein a first community-based organization provides a first community-based organization program of the plurality of community-based organization programs and a second community-based organization provides a second community-based organization program of the plurality of community-based organization programs;
establishing a second network connection to the electronic device through the network interface; and
returning the plurality of community-based organization programs that the patient qualifies for enrollment in through one or both of the single secure network connection and the second network connection.

9. The server of claim 8 further comprising;
receiving indications of needs of the patient; and
using the indications to select community-based organization programs that serve at least one of the needs such that comparing the patient attributes received through the single secure network connection to eligibility requirements is only performed on community-based organizations that serve at least one of the needs.

10. The server of claim 8 wherein returning the plurality of community-based organization programs comprises providing at least one control for requesting enrollment in one of the plurality of community-based organization programs.

11. The server of claim 10 further comprising:
sending the plurality of community-based organization programs that the patient qualifies for enrollment in through a third network connection to a second electronic device;
receiving an indication from the second electronic device that one of the plurality of community-based organization programs should be referred to the patient; and
in response, sending a referral for the one community-based organization program to the electronic device.

12. The server of claim 8 wherein each of the plurality of community-based organization programs has an end date and identifying the plurality of community-based organization programs comprises excluding community-based organization programs with an end date before a current date.

13. The server of claim 12 wherein for each community-based organization program, the end date is supplied by the community-based organization that provides the community-based organization program.

14. The server of claim 8 wherein each of the plurality of community-based organization programs has a maximum number of participants that can be enrolled in the program and wherein identifying the plurality of community-based organization programs comprises excluding community-based organization programs that have enrolled the respective maximum number of participants.

15. A method comprising:
receiving a single API call providing patient attributes of a patient;
comparing the patient attributes received through the single API call to eligibility requirements of multiple community-based organization programs to identify a plurality of community-based organization programs that the patient qualifies for enrollment in wherein a first community-based organization provides a first community-based organization program of the plurality of community-based organization programs and a second community-based organization provides a second community-based organization program of the plurality of community-based organization programs; and
returning the plurality of community-based organization programs that the patient qualifies for enrollment in through in response to a second API call.

16. The method of claim 15 wherein returning the plurality of community-based organization programs comprises providing at least one control for requesting enrollment in one of the plurality of community-based organization programs.

17. The method of claim 16 further comprising:
providing an indication to a community-based organization that the patient has requested to enroll in one of the community-based organization's programs; and
providing updates indicating the current status of the request to enroll in one of the community-based organization's programs.

18. The method of claim 15 wherein each of the plurality of community-based organization programs has an end date and identifying the plurality of community-based organization programs comprises excluding community-based organization programs with an end date before a current date.

19. The method of claim 18 wherein for each community-based organization program, the end date is supplied by the community-based organization that provides the community-based organization program.

20. The method of claim 15 wherein each of the plurality of community-based organization programs has a maximum number of participants that can be enrolled in the program and wherein identifying the plurality of community-based organization programs comprises excluding community-based organization programs that have enrolled the respective maximum number of participants.
